(19) United States
(12) Patent Application Publication
(10) Pub. No.: US 2013/0071927 A1
(43) Pub. Date: Mar. 21, 2013

(54) MEDIA AND METHODS FOR CELL CULTURE

(71) Applicants: David Lu, Riverwood (AU); Uli Schmidt, Rozelle (AU); Cara Bradley, Rozelle (AU); Sandra Lubitz, Hamburg (DE); Tomas Stojanov, North Narrabeen (AU)

(72) Inventors: David Lu, Riverwood (AU); Uli Schmidt, Rozelle (AU); Cara Bradley, Rozelle (AU); Sandra Lubitz, Hamburg (DE); Tomas Stojanov, North Narrabeen (AU)

(73) Assignee: Sydney IVF Limited, Sydney (AU)

(21) Appl. No.: 13/696,290

(22) PCT Filed: May 5, 2011
(86) PCT No.: PCT/AU2011/000512

§ 371 Date: Nov. 5, 2012

(30) Foreign Application Priority Data
May 5, 2010 (AU) 2010901922

(51) Int. Cl. C12N 5/0735

(52) U.S. Cl. CPC C12N 5/0606
USPC 435/366; 546/84; 435/325

(57) ABSTRACT

The present invention provides a cell passaging medium comprising at least one agent capable of detaching from a surface a cell that is culture in vitro on said surface, and a water-soluble polymer capable of protecting the detached cell. The present invention also provides a cell culturing medium comprising one or more cell culture protectants capable of protecting cells in culture. The present invention further relates to the use of said media in methods for culturing cells in vitro or for deriving monolayer cell cultures of mammalian stem cells.

### FIELD OF THE INVENTION

The present invention relates to compositions and methods for culturing cells. In particular, the invention relates to methods and media for use in monolayer cell culture and cell preparation.

The invention has been developed primarily for simple and efficient passaging and culturing of mammalian cells in monolayer culture, and will be described hereinafter with reference to this application. However, it will be appreciated that the invention is not limited to this particular field of use.

### BACKGROUND OF THE INVENTION

Any discussion of the prior art throughout the specification should in no way be considered as an admission that such prior art is widely known or forms part of the common general knowledge in the field.

### Stem Cells in Bioassays

Human pluripotent stem cells have great potential for applications in the pharmaceutical industry, clinical cell therapy and basic research. Applications in regenerative medicine and drug development for the high throughput testing of components require large numbers of cells—more than could be produced using traditional manual cell expansion techniques.

Cell-based assays are increasingly used in drug discovery and high-throughput screening due to the increased amount of information that can be obtained compared to biochemical assays. For cell-based assays small cultures are typically set up in microtiter plates with growth surfaces of 0.3 cm² or less. Current state-of the-art cell based assays use high-content analysis (HCA), a quantitative cell imaging technique which is a powerful tool for the fast and complex screening and analysis of effects that are measured in bioassays. HCA deals with each cell as a discrete experimental unit that dramatically increases the amount and quality of data obtained from an assay. Quantitative HCA analysis from conventional 3D pluripotent stem cell (PSC) cultures is possible, although considerably facilitated when cells are grown in a monolayer.

### Human Embryonic Stem Cell Culture

Traditionally, human embryonic stem cells (hESC) are derived and cultured on mouse embryonic fibroblasts (MEF) or human embryonic fibroblasts (HEF) as feeder cells.

These cultures can be maintained indefinitely by manual passaging by cutting out fragments of hESC colonies with a thin blade and transferring them onto a fresh feeder cell layer. This method generally does not show any apparent karyotypic or morphological changes or loss of pluripotency over time. An advantage of this method is that differentiated cell colony regions can be removed and only undifferentiated fragments are transferred. However, this technique may generate variable cluster size and result in inconsistent cell distribution, particularly for researchers less experienced with this method. The disadvantages of this method include:
1. high maintenance times;
2. difficulty to adapt the processes to automation;
3. growth pattern in multiple layers, making it sub-optimal for use in HCA;
4. propagation of cell clumps, not single cells, making uniform passaging and small volume culture in microtiter plates impossible, and
5. cell expansion is very tedious and only possible on a small scale.

In order to overcome some of these limitations, bulk passaging methods were explored. Bulk passaging methods either use calcium(II) chelating agents such as EDTA or proteolytic enzymes. Trypsin, collagenase IV and dispase were successfully used for passaging hESC on MEF or HEF feeder cells. These methods enable more consistent cell distribution, are less time consuming and allow the scaling of cultures to larger volumes and higher cell numbers.

However, the need for feeder cells introduces additional biological variability as well as time consuming steps for the maintenance of feeder cultures and the preparation of mitotically inactivated feeder plates.

The viability of hESC as single cells is generally poor. Therefore, mechanical and enzymatic passaging methods usually transfer hESC as clumps making them less useful for applications such as clonal selection, cell transfection or small volume cultures in microtiter plates which require a very even distribution of cells across the growth surface.

Three methods were recently described overcoming this limitation and allowing the passaging of hESC as single cells with improved viability. The first includes the addition of Y-27632 during trypsin/EDTA mediated cell dissociation. Y-27632 is an inhibitor of Rho-associated kinase (Rock), which blocks apoptosis via an as yet poorly understood pathway. This technique dramatically increases the cloning efficiency in both feeder and feeder-free cultures and hESC maintain their characteristic morphology of multi-layered colonies.

A second method describes the use of Accutase, a commercially available enzyme-based cell dissociation solution, for feeder-free, single cell hESC passaging on matrigel-coated tissue culture vessels. The enzyme mix contained in Accutase does not result in poor single cell viability observed with other enzymes. hESC maintained by this method remain pluripotent but eventually change their morphology to cell monolayers.

A third method makes use of the observation that single hESC show improved viability when plated at a very high density. Cells are dislodged from MEF or HEF feeders as single cells using trypsin/EDTA and plated at high density onto matrigel-coated tissue culture dishes. After this initial adaptation step hESC can be subsequently passaged as single cells with trypsin/EDTA at standard density but with improved clonal viability.

Notwithstanding these advances, all bulk passaging and feeder-free hESC culture methods raise questions about the long-term quality of the cells particularly in terms of their karyotypic stability. hESC lines propagated by manual passaging generally retain normal karyotypes for more than 100 passages whereas bulk methods frequently, but not always, acquire abnormalities after 20-40 passages. The reason for karyotypic changes is most likely a gradual adaptation of the cells to culture conditions, whereby certain mutations and chromosomal defects provide growth advantages to altered subpopulations of cells. This is most likely not caused solely by bulk passaging techniques but may involve other stresses such as cell density. Also, for a given mutation rate the probability of genetic changes occurring in a group of cells depends on the population size, and as manual passaging techniques are generally restricted to a small scale, mutations may be less likely to occur for that reason.

A further limitation of the above methods is that it is generally not easy to switch between different culturing methods without a period that allows the cells to reach their optimal growth characteristics under the new conditions. This often causes significant delays particularly when small scale manually maintained hESC cultures have to be expanded for downstream applications or even for basic cell line characterization tests which require higher cell numbers.

Thus, there is a need for passaging methods, passaging media and culture media which ameliorate at least some of the limitations of current systems described above, by combining particular culture techniques and media components for each step in the cell supply chain and allowing rapid switching between these techniques without any adaptation and lag periods.

### SUMMARY OF THE INVENTION

According to a first aspect, the present invention provides a cell passaging medium comprising at least one agent capable of detaching from a surface a cell that is cultured in vitro on said surface, and a water-soluble polymer capable of protecting the detached cell.

Preferably, the agent capable of detaching cells from a surface is a metal ion chelating agent and/or a proteolytic enzyme. However it will be understood that a combination of such agents may also be used, depending on the needs of the culture system used. Any agent, or combinations thereof, capable of detaching cells from a surface on which they grow may be advantageously employed in the media and methods of the present invention.

Preferably, where a combination of a metal ion chelating agent and a proteolytic enzyme is employed, a ratio of high chelator to low enzyme is used, e.g. 0.5% trypsin+5 mM EDTA.

Examples of suitable metal chelating agents are those that bind divalent metal ions and can be selected from EGTA, EDTA, crown ethers or cryptands. Examples of suitable proteolytic agents are collagenase, trypsin, dispase, accutase, from natural or recombinant sources or combinations of two or more proteolytic agents.

The surface on which cells are cultured is preferably a solid surface such as for example a glass or plastic culture plate, flask, dish, microtiter plate, chamber slide, coverslip or similar utensil.

When grown on a solid surface cells may be cultured and maintained on feeder layers such as fibroblast feeder layers, or the surface may be coated with agents such as collagen or matrigel. However, this is not always necessary when using the passaging/culture media and methods of the present invention. The cells may be in a short-term primary cell culture or a long-term culture of an immortal cell line. Preferably, the cells are stem cells and more preferably they are pluripotent stem cells. Even more preferred are pluripotent human embryonic stem cells or human induced pluripotent stem cells. The cells are preferably cultured on a solid surface.

As is known by those of skill in the relevant art the viability of cells and particularly the viability of human embryonic or induced pluripotent stem cells, cultured in vitro after single cell dissociation is significantly reduced resulting in low cell numbers and slow cell expansion. The passaging medium of the present invention, which makes use of a water soluble polymer, is able to enhance/preserve cell viability during passaging.

The water-soluble polymer can be advantageously selected from a range of synthetic or natural organic polymers, for example, gelatin, polyvinylpyrrolidone (PVP), polyethylene glycol (PEG), agarose, dextran, polypeptides, polysaccharides or polynucleotides.

The term “water-soluble polymer” as used in the context of the present invention is intended to encompass any polymer that has the ability to protect the cells and maintain the cells' status once they are in suspension following passaging and before they are cultured again on a substrate or in suspension.

According to a second aspect, the present invention provides a method of passaging a cell cultured on a surface, comprising detaching the cell from said surface using a cell passaging medium according to the first aspect.

The cell passaging media, culture media and methods of the present invention are capable of maintaining the cell status of a cell during a passage and subsequent culture.

Thus, according to a third aspect, the present invention also provides a method of maintaining the cell status between passages of a cell cultured on a first surface, comprising the steps of:

(i) detaching the cell from the first surface using a cell passaging medium according to the first aspect, and
(ii) culturing said detached cell on a second surface.

According to a fourth aspect, the present invention also provides use of a cell passaging medium according to the first aspect to maintain the cell surface status of the cell during a passage.

In another embodiment of the present invention the cell viability after dissociation to single cells is increased by the use of a protectant which is present in the subsequent culture medium.

Thus, according to a fifth aspect the present invention provides a cell culturing medium comprising one or more cell culture protectants capable of protecting cells in culture.

Cell culture protectants may be chosen from agents that modulate apoptosis and/or apoptotic pathways, such as for example caspase inhibitors, p53 inhibitors or agents that modulate anoikis or the myosin pathway, such as for example the Rho kinase inhibitors, Rho associated coiled-coil kinase (ROCK) inhibitors, MYPT1 inhibitors, MRLC inhibitors or myosin II inhibitors. Cell culture protectants increase the viability of single cells after passaging. The protectants do not otherwise change or affect the cell status. Cell culture protectants may also modify the cell's biomechanical and/or adhesion properties to facilitate growth in a monolayer on a substrate without otherwise changing or affecting the cell status. Such a reagent may also be referred to herein as “modulator” or “protectant/modulator”. Once the cells proliferate and form colonies the protectants may be dispensable and hence may be removed from the culturing medium if desired. Of course it will be understood that the protectants may remain in the culture medium at all times. Examples of suitable protectants include the caspase inhibitors Boc-Asp(OMe)-Fluoromethylketone or Quinoline-Val-Asp-Difluorophenoxymethylketone (OPH109), the p53 inhibitors pifithrin-α or cyclic pifithrin, the ROCK inhibitors Y27632, fasudil or H1152, Rho inhibitors such as C3, Rhodblock 1a or Rhodblock 3 and the myosin II inhibitors such as blebbistatin or N-benzyl-p-toluenesulfonamide (BTS).

Accordingly, the present invention provides, in a sixth aspect, a cell culturing medium comprising one or more cell culture protectants capable of maintaining the cells in a monolayer culture on a surface.

According to a seventh aspect, the present invention o provides a method of continuously maintaining a culture of cells growing on a first surface, comprising repeated steps of:

(i) detaching the cell from the first surface, and
(ii) culturing said detached cell on a second surface in a culture medium according to the fifth aspect.

The cells may be detached from the first surface using conventional passaging media known in the art or may utilise the passaging medium of the present invention, as described in the first aspect.

In one embodiment the passaging media, culture media and methods of the present invention are used to expand cells. Preferably, the cells are expanded at least 10 times per passage.

It will be appreciated by those of skill in the relevant art that subsequent downstream culturing of the passaged cell may be achieved by traditional cell culture techniques known to those of skill in the art. However, to achieve a true monolayer cell culture, in an embodiment of the present invention the passaged cells, which may be passaged using conventional media, are cultured using a medium that contains cell culture protectants which modulate cells in a way that enables them to grow in a monolayer on a substrate, preventing the common multi-layer growth pattern.

According to an eighth aspect, the present invention provides a method of culturing cells in a monolayer on a surface, comprising the step of culturing the cells using a cell culture medium according to the seventh aspect.

It is a particular advantage of the media and methods of the present invention that cells, particularly hESC and human induced pluripotent stem cells, can be cultured as monolayers in a feeder-free system. Such monolayers can be used in further applications such as pharmaceutical research and the like, while ameliorating the biological variability and the time consuming maintenance steps inherent in feeder cell cultures.

According to a ninth aspect, the present invention provides a method of culturing a cell in a feeder-free monolayer on a surface, comprising the step of culturing the cell on the surface using a cell culture medium according to the seventh aspect.

Of course, it will be appreciated by those of skill in the art that other cell culture modulators may be added to the cell culture medium according to the invention.

According to a tenth aspect, the present invention provides a method of passaging and culturing cells, comprising the steps of:
i. detaching the cells from a first surface; and
ii. culturing the detached cells on a second surface using a cell culture medium according to the seventh aspect.

It will be understood that the cells may be detached from the first surface using conventional passaging media known in the art or may employ the passaging medium of the present invention as described in the first aspect. In one embodiment the passaging media, culture media and methods of the present invention enable rapid switching between long-term cell culture and short-term expanded monolayer cell culture due to the cell-protective and modulating effects provided by the passaging medium and/or the culture medium.

Thus, according to an eleventh aspect, the present invention provides a method of passaging cells between long-term cell culture maintained on a surface and short-term expanded monolayer cell culture, comprising the step of detaching cells from said surface and culturing said detached cells in a monolayer using a culture medium according to the seventh aspect.

It will be understood that in this aspect also the cells may be detached from the first surface using conventional passaging media known in the art or may employ the passaging medium of the present invention as described in the first aspect.

The first and/or second surface may be a surface coated with cell growth and/or cell attachment agents or compositions such as matrigel. One of the substrates or coatings may be feeder cell layer, such as a fibroblast feeder layer. Alternatively, the first and or/second surface may be uncoated tissue culture treated or non-treated plastic surfaces.

The culture media of the present invention may include both a protectant and a modulator. Advantageously, a single reagent may be both a protectant and a modulator.

Thus, in one embodiment the functions of the protectant and the modulator are combined within the same reagent. An example of a combined protectant/modulator is the ROCK inhibitor Y27632.

Accordingly, the present invention provides, in a twelfth aspect, a cell culturing medium comprising one or more cell culture protectants and one or more cell culture modulators capable of maintaining the cells in a monolayer culture on a substrate.

According to a thirteenth aspect, the present invention also provides a method of maintaining the cell status between a passage of a cell cultured on a first substrate, comprising the steps of:

(i) detaching the cell from the first surface and
(ii) culturing said detached cell on a second surface using a cell culture medium according to the seventh or twelfth aspects.

It is yet a further advantage that the invention provides a method of maintaining the cell status between a passage of a cell between long-term cell culture maintained on a first surface and short-term expanded monolayer cell culture on a second surface.

According to a fourteenth aspect, the present invention also provides a method of maintaining the cell status between a passage of a cell between long-term cell culture maintained on a first surface and short-term expanded monolayer cell culture on a second surface, comprising the steps of:

(i) detaching the cell from the first surface, and
(ii) culturing said detached cell on a second surface using a cell culture medium according to the seventh or twelfth aspects.

The term “cell status” as used in the context of the present invention is intended to encompass the main characteristics and properties of the cell including the molecular composition of the cell which typify said characteristics and properties. This may include cell surface markers, transcription factors, messenger RNAs, micro RNAs and epigenetic modifications. The term may also encompass the composition of the cell's membrane lipid bilayer, composition of membrane bound or anchored proteins, cell surface markers and other characteristics which may be damaged or lost on passaging from one culture to another, and in particular during long-term culture and frequent passaging. The maintenance of the cell status also contributes to increased viability and utility of the cultured cells. For example the main characteristic of human embryonic stem cells or human induced pluripotent stem cells is their pluripotency which is indicated by the presence of cell surface markers such as SSEA-4 or Tra-1-60 or transcription factors such as Nanog or Oct-3/4. However, the cell's overall composition may vary without affecting their typical composition. For example, the composition of cell adhesion proteins may change depending on the type substrate the cells are cultured on while the composition typifying pluripotency remains unaffected. It will however be appreciated by those skilled in the art that for some applications it may be desirable to change the cell status, for example by differentiating pluripotent stem cells to somatic cells. The current invention easily allows this by using a culture medium that facilitates the change and by adding the protectant and/or modulator to this medium.

It will be understood from the disclosure provided herein that the passaging media, the culture media with protectants, and the culture media with both protectants and modulators, may be used in any combination to achieve various desired effects and advantages, or may be all combined to optimise the culture conditions.

Thus, according to a fifteenth aspect, the present invention provides a method of passaging and culturing cells, comprising the steps of
i. detaching the cells from a first surface using the passaging medium according to the first aspect; and
ii. culturing the detached cells on a second surface using a cell culture medium according to any one of fifth, seventh or twelfth aspects.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### BRIEF DESCRIPTION OF THE DRAWINGS

FIG. 1 shows a growth curve of hESC passaged by gelatin/EGTA. SIVF001 hESC were passaged 24 times using gelatin/EGTA and plated into 12 well plates for cell number analysis on days 4-7. Error bars are standard deviation of triplicate cell culture results;

FIG. 2 shows passaging of hESC SIVF001 using different water-soluble polymers. Cells were passaged and plated into 6-well plates for cell number analysis. After 7 days hESC colonies were clearly visible and cells were harvested and counted.

FIG. 3 shows hESC (A: SIVF006; B: SIVF022) grown as a feeder cell-free monolayer culture on collagen I using combinations of protectants and modulators. Cells were plated in 96-well plates with the additives as shown. After 4 days the cells were fixed, stained for the markers Oct-3/4, CD29, SSEA-4, Tra-1-81 and analysed by HCA. Bars indicate averages from 3 wells with standard deviations as error bars.

FIG. 4 shows a growth curve of hESC grown as a feeder cell-free monolayer. hESC SIVF019 were plated onto collagen I coated or uncoated wells, with the protectant/modulator Y-27632;

FIG. 5 shows the expression of pluripotency markers in different hESC lines grown in a feeder-free monolayer. Note that Y-27632 was excluded from the medium 24 hrs after plating;

FIG. 6 shows expansion of hESC in a monolayer. Note that the stock of SIVF019 hESC (A) were collagenase passaged, whereas SIVF002, SIVF006 and SIVF021 (B) were manually passaged. Y-27632 was removed from the medium the day after passaging. All cells counts are viable cells only;

FIG. 7 shows the expression of pluripotency markers in hESC after passaging of cells as a monolayer. Note that the SIVF019 hESC used for this experiment are the same as shown in FIG. 6;

FIG. 8 shows the growth and expression of pluripotency markers in feeder-free SIVF019 hESC monolayer cultured on different surfaces;

FIG. 9 shows the transfection of a monolayer of hESC. SIVF019 hESC were transfected with 3 plasmids at 2 different concentrations.

### PREFERRED EMBODIMENT OF THE INVENTION

The method of the invention is advantageous as it permits passaging between the main modes of pluripotent stem cell culture, i.e. (i) maintenance of PSCs on human feeder cells (feeder culture); (ii) feeder-free expansion of PSCs (feeder-free culture).

In particular, the feeder-free culture method uniquely results in monolayer growth morphology of PSCs which is advantageous for cellular imaging and high-content analysis.

In addition, pluripotent stem cells can easily be switched between different culturing modes without the need for adaptation steps, therefore providing flexibility and the ability to rapidly respond to changing culturing requirements. PSCs are converted into a single cell suspension by disrupting calcium-dependent cell-cell junctions using a chelating agent with or without the addition of suitable proteolytic enzymes or other agents capable of detaching cells from a solid surface. The use of single-cell suspensions results in an even cell distribution across the culture surface which is essential for setting up small volume cultures (e.g. microtiter plates) as used in cell-based medium to high-throughput screening assays.

In order to increase the viability of the cells during or after dissociation to a single-cell suspension a water-soluble polymer in the passaging solution or a cell protectant in the subsequent culture medium provide protection of the cells. These fast and simple methods are characterised by high split ratios, high cellular viability despite single-cell passaging and a stable cellular karyotype in medium to long-term culture. PSCs maintained in the manual system (LT-M) described above, are easily transferred to the LT-CP system. Advantageously, the media and methods according to the invention enable the passaging and switching of cells into different cultures to be fully automated.

For many applications, including cell differentiation, bioassays and most hESC quality control tests, it is desirable to culture hESC in the absence of feeder cells and ideally as a monolayer rather than the typical morphology of three-dimensional, multilayer colonies. The use of a passaging medium and a culture medium containing a cell protectant/modulator according to the invention permits the passaging of single cells and their subsequent culture in a monolayer while maintaining their cell status without the need for a biological matrix and feeder-cell support.

It is a further advantage of the media and methods of the invention that PSCs maintained with both the manual system and the feeder-based culture system described above can be transferred directly to the feeder-free system without any adaptation steps. The feeder-free system is also fully automatable.

A preferred embodiment of the invention will now be described, by way of example only, with reference to the accompanying examples

### EXAMPLES

### Example 1

### Passaging of hESC Using Gelatin/EGTA

hESC used for these experiments were derived as described previously (Peura et al., 2008). In brief, blastocyst stage embryos were plated onto gelatin-coated tissue culture dishes containing mitomycin C-inactivated human foetal fibroblasts (hereafter referred to as feeder cells; ATCC) in KO-DMEM with 20% Knockout Serum Replacement (KSR), 2 mM glutamine, 50 U/ml penicillin, 50 mg/ml streptomycin, 1×MEM-amino acids, 0.1 mM β-mercaptoethanol (all Invitrogen), hereafter referred to as KSR medium, and 20 ng/ml bFGF (Sigma). Cells were incubated at 37° C./5% C0₂/5% O₂, with outgrowths from the inner cell mass expanded by manual passaging and cultured as described above, with the exception that 4 ng/ml bFGF was used in the KSR medium. Outgrowths were karyotyped and confirmed as pluripotent hESC lines by evaluation of pluripotency markers and the ability to form the 3 germ layers in teratoma experiments using SCID mice. The hESC lines used in examples are listed in Table 1.

For the rapid and simplified expansion of hESC cultures, hESC were passaged with gelatin/EGTA solution. The solution was made by dissolving 0.5% gelatin (Sigma #01890) in phosphate buffered saline free of magnesium and calcium (hereafter refer to as PBS) (Invitrogen 14190-144), and addition of EGTA (Sigma #E0396) to a final concentration of 2 mM, prior to autoclaving. The cultures were washed with PBS twice and incubated with a sufficient volume of gelatin/EGTA to cover the entire growth surface area for 20 mins at 37° C. Colonies were dissociated into single cells by repeated pipetting and passed through a 35 μm cell strainer (BD Falcon, #352235) to remove any feeder layer carried over. The single cells were diluted using KSR medium and centrifuged at 250×g for 4 mins. The cell pellet was resuspended in KSR medium and cell number determined using a cell counter (NucleoCounter, Chemometric). Cells were then replated into new cell culture vessels with prepared feeder cells at approximated 5×10³/cm² in KSR medium with 4 ng/ml bFGF and incubated at 37° C./5% O₂/5% CO₂. The medium was changed every 2^{nd} day, with cells passaged once weekly, with approximately a 1:10 split ratio (−5×10⁴/cm²) used.

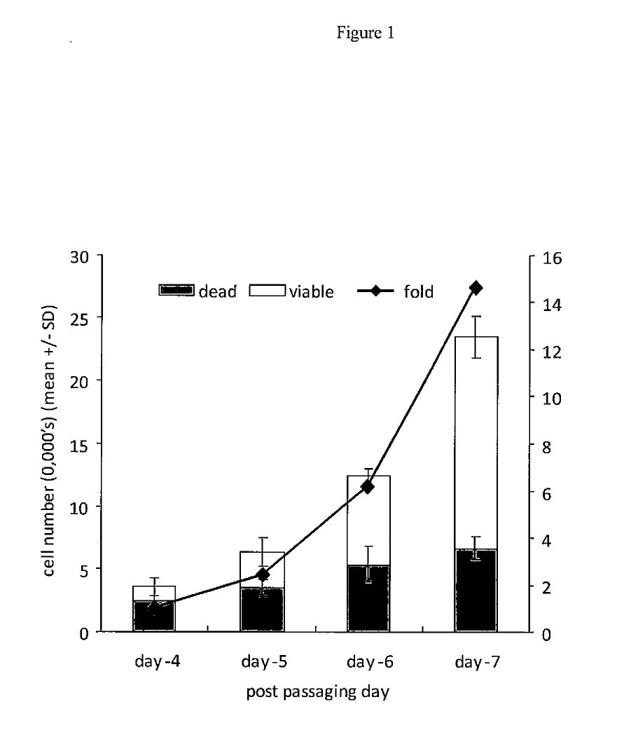

The growth of SIVF001 hESC passaged 15 times using gelatin/EGTA was monitored by light microscopy over 7 days. Small colonies became clearly visible 4 days after passage, and expanded considerably in size by day 7. The hESC showed typical morphology of multilayered hESC colonies grown on feeders. The growth rate of hESC after passaging 24 times using gelatin/EGTA was monitored by counting of cells at days 4-7 post-plating (FIG. 1). A 15-fold expansion in cell number from day 4 to day 7 was observed, with the doubling time calculated as 19.5 hrs. This growth rate is similar to reported growth rates for undifferentiated parts of hESC colonies further indicating the preservation of the typical characteristics of pluripotent hESC.

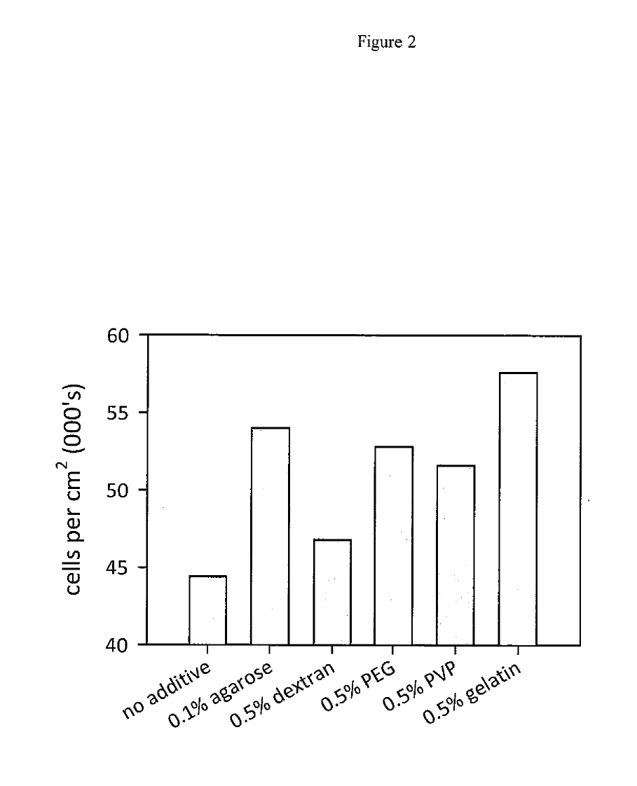

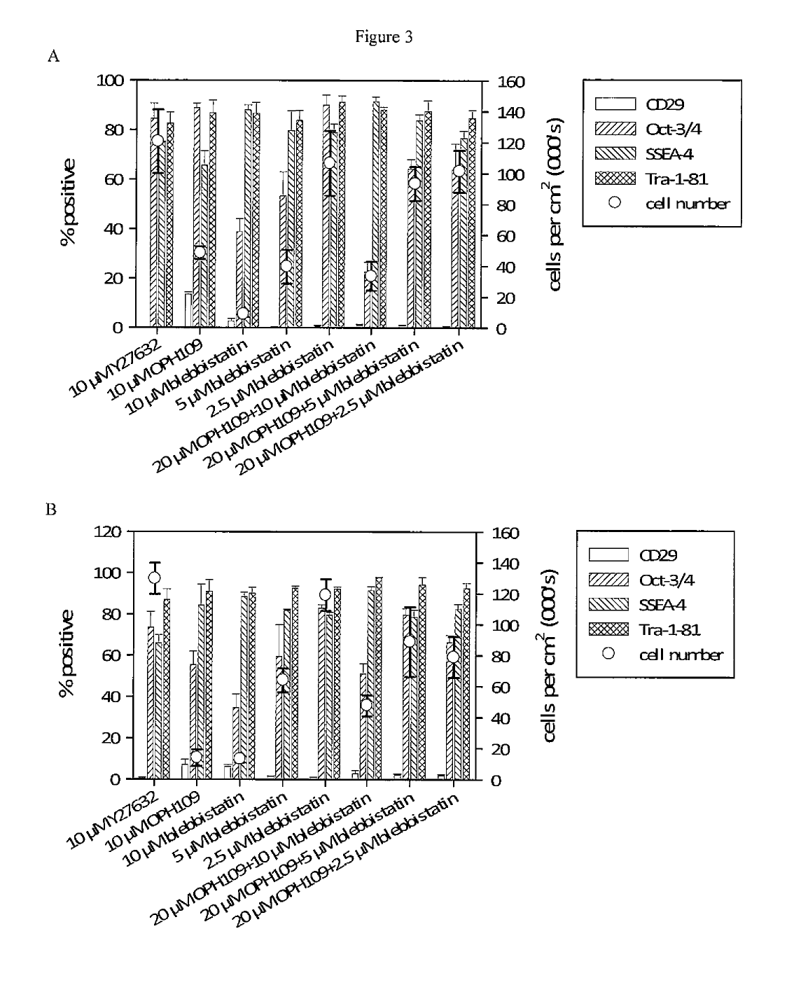

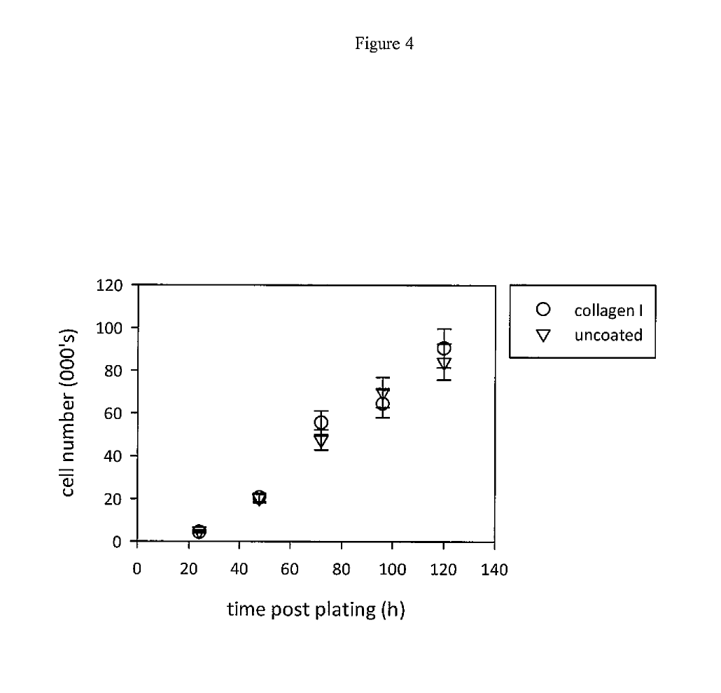

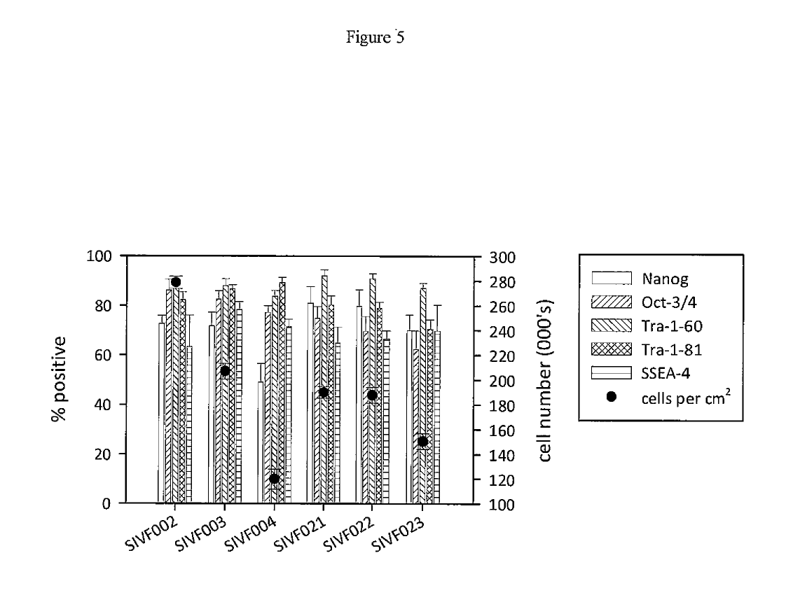

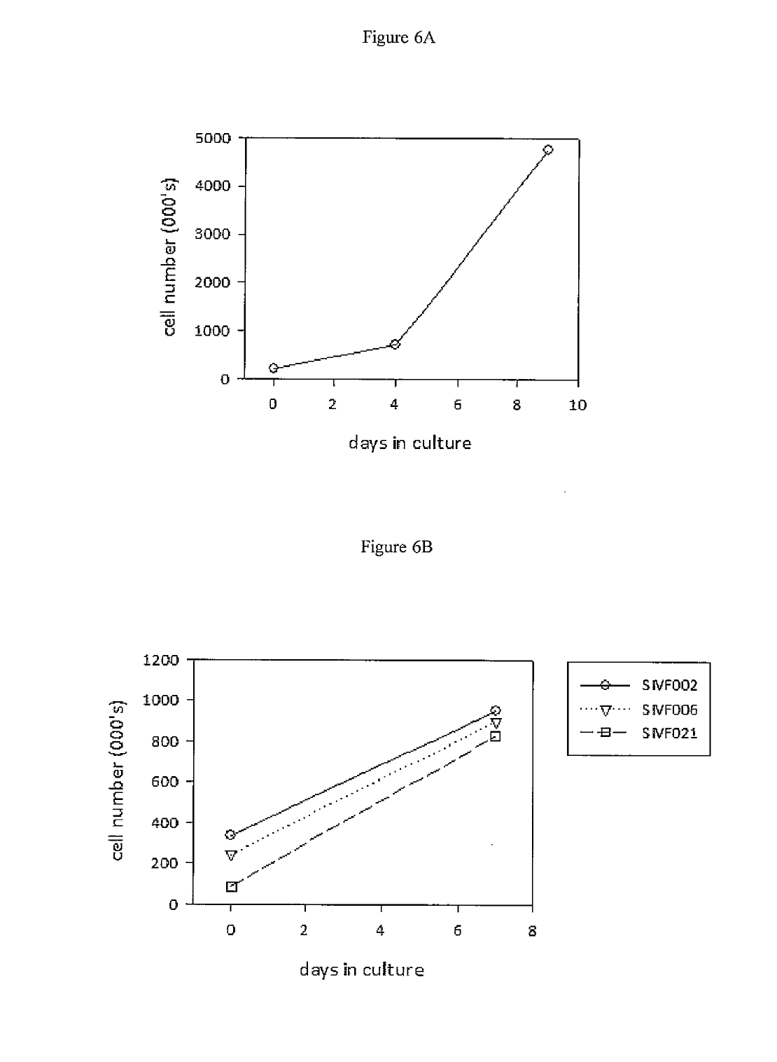

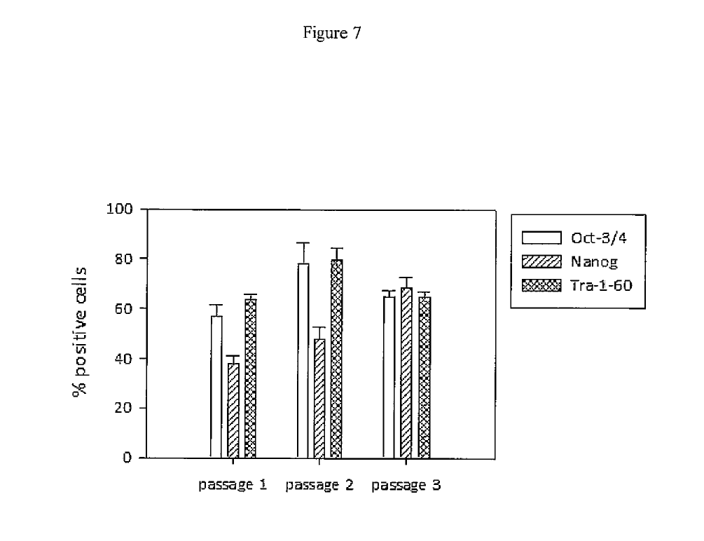

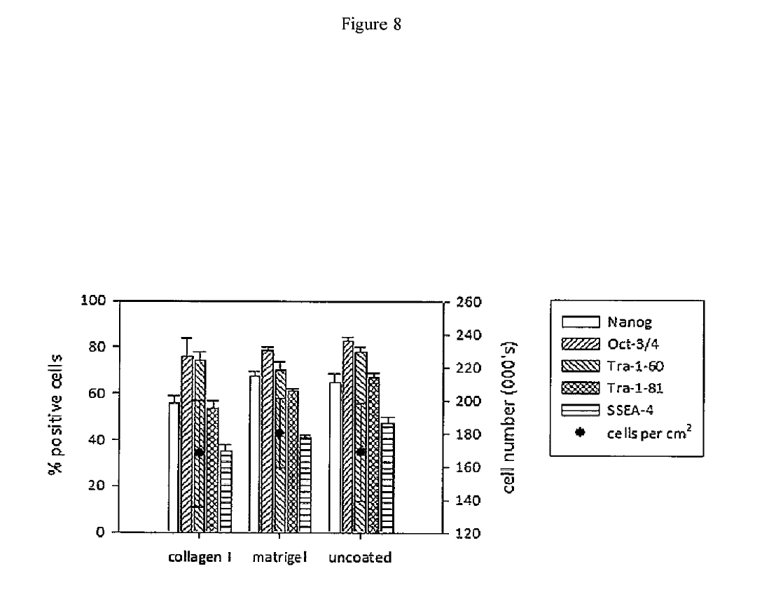

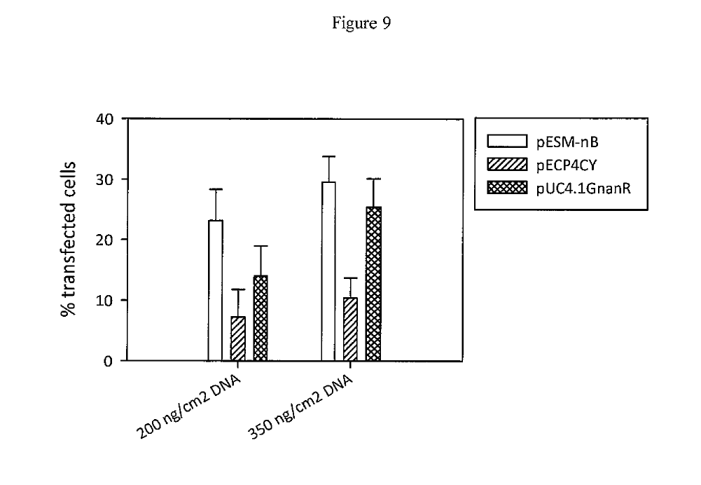

TABLE 1
hESC lines used in the described examples. Pluripotency “in
vitro” refers to immunohistochemical analysis of pluripotency
stem cell markers and “in vivo” refers to teratoma experiments.

| | | Pluripotency | | |
|---|---|---|---|---|
| Cell line ID | Karyotype | In vitro | In vivo | Figure |
| SIVF001 | 46, XX | Yes | Yes | FIG. 1, FIG. 2, |
| | | | | FIG. 3, FIG. 4, |
| | | | | FIG. 5, Table 2 |
| SIVF002 | 46, XY | Yes | Yes | FIG. 9, FIG. 12 |
| SIVF003 | 47, XX, | Yes | In progress | FIG. 9 |
| | +16 | | | |
| SIVF004 | 46, XX | Yes | Yes | FIG. 9 |
| SIVF006 | 46, XX | Yes | Yes | FIG. 3, FIG. 12 |
| SIVF019 | 46, XX | Yes | In progress | FIG. 6, FIG. 7, |
| | | | | FIG. 10, FIG. 11, |
| | | | | FIG. 12, FIG. 13, |
| | | | | FIG. 14, FIG. 15, |
| | | | | FIG. 16, Table 2 |
| SIVF021 | 47, XY, | Yes | In progress | FIG. 9, FIG. 12 |
| | +21 | | | |
| SIVF022 | 46, XY | Yes | In progress | FIG. 3, FIG. 9 |
| SIVF023 | 46, XY | Yes | In progress | FIG. 9 |
| SIVF025 | 46, XY | Yes | In progress | FIG. 8 |
| SIVF028 | 46, XX | Yes | In progress | FIG. 8 |

### Example 2

### Characterization of hESC Passaged Using Gelatin/EGTA

To determine if hESC lines passaged using gelatin/EGTA maintain the hallmark characteristics of stem cells, the karyotype and expression of pluripotency markers after multiple passages was examined.

hESC were passaged using gelatin/EGTA as described in Example 1. Cells were karyotyped as previously described (Peura et al., 2008). For enrichment of cells in M phase, outgrowths were incubated with either 0.22 ng/ml colcemid (KaryoMAX) and 37.5 g/ml BrdU for 17-19 hrs or 5 ng/ml colcemid for 2.5 hrs. Single cells were subsequently obtained using Non-enzymatic Cell Dissociation Solution (Sigma) and metaphase spreads prepared for G-banding. Karyotyping revealed SIVF001 hESC at gelatin/EGTA passage 13, 20 and 33, as well as SIVF019 cells at passage 12, were cytogenetically normal (Table 2).

SIVF001 hESC passaged 27 times using gelatin/EGTA were assessed for the expression of pluripotency markers by immunohistochemistry. The cells were plated into 96 well optical bottom plates (BD Falcon #353219) at a density of ˜3.7×10³/cm² and were grown for 6 days. The cells were washed briefly with PBS with magnesium and calcium (PBS+), fixed with 4% paraformaldehyde for 15 mins and washed 3 times with PBS+. Wells were incubated with primary antibodies Oct4-Alexa 488, SSEA4-Alexa 488, Tra160-Alexa 555, Tra181-Alexa 555 (all BD Falcon) or Nanog (Santa Cruz), and 0.25% Triton X-100 (Sigma) in KSR medium for 1 hour, then washed 3 times with PBS+. Wells stained with anti-Nanog primary antibody were incubated with the secondary antibody donkey anti-goat IgG-Alexa 488 (Invitrogen) in KSR medium for 1 hour then washed 3 times with PBS+. Wells were counterstained with DRAQ5 (Biostatus) and assessed by fluorescence microscopy. The hESC colonies stained positive for all pluripotency markers while the feeder cells remained unstained. These experiment revealed that hESC passaged using gelatin/EGTA retain their expression of pluripotency markers.

TABLE 2
Karyotype of hESC after multiple passages using
gelatin/EGTA. hESC were maintained by manual
passaging prior to using gelatin/EGTA.

Passages

| Cell line | total | # gelatin/EGTA | Karyotype |
|---|---|---|---|
| SIVF001 | 23 | 13 | 46, XX |
| SIVF001 | 30 | 20 | 46, XX |
| SIVF001 | 43 | 33 | 46, XX |
| SIVF019 | 60 | 12 | 46, XX |

### Example 3

### Testing of Different Water-Soluble Polymers for hESC Passaging

To determine if other water-soluble polymers are suitable for hESC passaging, hESC were passaged using EGTA combined with either agarose, dextran, PEG, PVP or gelatin.

Single cells were generated using the same method as described in Example 1, with the exception of different water-soluble polymers used in place of gelatin, being either 0.1% agarose (Sigma #A2576), 0.5% dextran (Sigma #00269), 0.5% PEG (Sigma #P3015) or 0.5% PVP (Sigma #P5288). All water-soluble polymers were dissolved in PBS containing 2 mM EGTA and autoclaved, hESC used in the experiment were SIVF001 cells which had been passaged 30 times using the gelatin/EGTA method, seeded into 6 well plates (FIG. 5A). These cells were then passaged 3 times with a split ratio of 1:10 using different water-soluble polymers/EGTA solutions. Significantly more hESC colonies were observed in the presence of water soluble polymers. After 7 days in culture the cells were harvested and counted. As seen in FIG. 2, passaging of cells with a water-soluble polymer is critical for hESC survival. The use of water-soluble polymers other than gelatin also improved cell survival over EGTA alone, although somewhat less efficiently than gelatin/EGTA.

### Example 4

### Expansion of hESC Using Protectants in the Culture Medium

A simplified protocol for a rapid transfer and expansion method of hESC previously maintained manually on feeder layer cells in organ culture dishes was investigated.

Organ culture dishes with various hESC lines (Table 1) were washed once with 1 ml PBS and then dislodged by adding ˜0.2 ml 0.05% trypsin/5 mM EDTA and incubating at 37° C. for 10 mins. After the incubation 1 ml DMEM medium containing 10% FBS was added to inhibit the trypsin, and a single cell suspension was obtained by vigorous pipetting. The cells were counted, pelleted by centrifugation at 250×g for 4 min and resuspended in 5 ml KSR medium containing 20 ng/ml bFGF and 20 μM OPH109, a potent, cell permeable inhibitor of several caspases. The cell suspension was then transferred to a T25 culture flask containing fresh, mitomycin C treated human embryonic fibroblast feeder layer cells and incubated at 37° C., 5% CO₂ and 5% O₂. The medium was changed after 2 days (and every 2 days thereafter) using KSR medium plus 4 ng/ml bFGF. After 3-4 days in culture a dense pattern of hESC colonies became visible which showed the typical morphology of small cells with a low cytoplasmic to nuclear ratio growing in multilayered colonies. Over the course of 8-10 days these colonies continued growing in size. In contrast, control cultures set up in the same way but without OPH109 in the initial culture medium only contained very few hESC colonies, in concordance with the reported poor viability of hESC after single cell dissociation. When observing the cultures by light microscopy and taking 10 random images using a 10× objective, colonies were observed in 6 out of the 10 images for cultures containing OPH109 and only 1 out of 10 for the control cultures without OPH109.

After 8-10 days the cells were harvested by preparing a single cell suspension using trypsin/EDTA as described above. Table 3 list the numbers of cells obtained for various cell lines, indicating a 10-15 fold expansion in a single step. The cells could be used for subsequent experiments or further expansion by plating 10,000 cells/cm² into culture vessels with fresh feeder layer cells using KSR medium plus 20 ng/ml bFGF and 20 μM OPH109 as the initial culture medium. Cell lines were maintained in that way for >10 passages with a 10-15-fold expansion per passage while maintaining their typical morphology, the expression of pluripotency markers and a stable karyotype.

In another experiment, single cell suspensions were prepared from hESC cells grown in organ culture dishes using trypsin/EDTA as described above. Cell were then resuspended in 5 ml KSR medium containing 20 ng/ml bFGF and 2.5 μM blebbistatin, a potent, cell permeable inhibitor of myosin II. The cell suspension was then transferred to a T25 culture flask containing fresh, mitomycin C treated human embryonic fibroblast feeder layer cells and incubated at 37° C., 5% CO₂ and 5% O₂. The medium was changed after 2 days (and every 2 days thereafter) using KSR medium plus 4 ng/ml bFGF. Again, after 3-4 days in culture a dense pattern of hESC colonies became visible which also showed the typical morphology of small cells with a low cytoplasmic to nuclear ratio growing in multilayered colonies. Colonies continued to grow in size and density over 8-10 days until the cultures were passaged again for cell counting (summarised in Table 4) and replating of 2,000-10,000 cells/cm² into culture vessels with fresh feeder layer cells using KSR medium plus 20 ng/ml bFGF and 2.5 μM blebbistatin as the initial culture medium. Using this method cells were expanded for >10 passages, while maintaining their pluripotency and normal karyotypes. Blebbistatin was found to be a more powerful protectant than OPH109, allowing lower initial plating densities and greater expansion.

Performing similar experiments using the ROCK inhibitor Y27632 as a protectant in the culture medium also resulted in dramatically increased cell viability after dissociation into single cell suspensions. The morphology of the colonies was altered and found to be flat monolayers of small cells with high nuclear to cytoplasmic ratios.

This example demonstrates the usefulness of protectants to increase the viability of single cell dissociations allowing the rapid switching from manual to bulk passaging without adaptation steps and rapid long term expansion of hESC lines.

TABLE 3
Numbers of viable cells transferred from organ culture dishes to T25
culture flasks using OPH109 as a protectant in the culture medium.

| | Viable cells in | Viable cells after |
|---|---|---|
| Cell line | organ culture dish | 1 expansion step |
| SIVF021 | 2.19 × 10⁵ cells | 3.00 × 10⁶ cells |
| SIVF006 | 1.23 × 10⁵ cells | 1.97 × 10⁶ cells |
| SIVF022 | 3.99 × 10⁵ cells | 4.40 × 10⁶ cells |
| SIVF019 | Not determined | 3.1 × 10⁶ cells |

TABLE 4
Numbers of viable cells transferred from organ
culture dishes to T25 culture flasks using blebbistatin
as a protectant in the culture medium.

| | Viable cells in | Viable cells after |
|---|---|---|
| Cell line | organ culture dish | 1 expansion step |
| SIVF002 | 5.7 × 10⁴ cells | 1.5 × 10⁶ cells |
| SIVF019 | Exp 1: 6.6 × 10⁴ cells | 3.4 × 10⁶ cells |
| | Exp 2: 2.5 × 10⁵ cells | 2.4 × 10⁶ cells |
| | Exp 3: 2.5 × 10⁵ cells | 3.4 × 10⁶ cells |
| SIVF024 | 2.6 × 10⁵ cells | 1.6 × 10⁶ cells |
| SIVF048 | 2.3 × 10⁵ cells | 2.0 × 10⁶ cells |

TABLE 5
Long term expansion of hESC lines passaged as single cells and using
protectants in the culture medium maintained their karyotype.

| | Number of passages as single | Karyotype - no |
|---|---|---|
| | cells and using protectants | abnormalities |
| Cell line | in the culture medium | were detected |
| SIVF006 | 14 | 46, XX |
| SIVF022 | 15 | 46, XY |
| SIVF023 | 10 | 46, XY |
| SIVF002 | 10 | 46, XY |
| SIVF005 | 10 | 46, XY |
| SIVF007 | 11 | 46, XX |
| SIVF017 | 10 | 46, XY |

### Example 5

### Establishment of a Feeder Layer-Free Monolayer hESC Culture

Methods for the feeder free culture of hESC were established. Following the dissociation into single cells using trypsin/EDTA the plating of hESC in the presence of protectants/modulators was tested.

hESC used in these experiments are described in Table 1 and were maintained either as described in Example 4 or by Collagenase passaging (Invitrogen). The collagenase passaged hESC were also cultured on an inactivated feeder cell layer with KSR medium and 4 ng/ml bFGF.

A single cell suspension was prepared similar to Example 4 by first washing a T25 flask with 5 ml PBS followed by trypsinising the cells with 2 ml 0.05% trypsin/5 mM EDTA, incubating at 37° C. for 10 mins and resuspending in 10 ml DMEM medium containing 10% FBS with vigorous pipetting. Cell number and viability was determined using the NucleoCounter and an appropriate number of cells centrifuged at 250×g for 4 mins prior to resuspension in conditioned KSR medium with 20 ng/ml bFGF plus additives. Conditioned KSR medium was produced by incubation of KSR medium on mitomycin-C treated human foetal fibroblasts for 48 hrs prior to harvest and storage at −20° C. As protectants/modulators the following additives were tested:
1. 10 μM Y-27632
2. 20 μM OPH109
3. 2.5-10 μM blebbistatin
4. 20 μM OPH109+2.5-10 μM blebbistatin

Cells were plated in collagen I (Cell Science and Technologies) coated 96 well optical bottom plates (BD) at a density of 2×10⁴ cells/cm², and incubated at 37° C./5% CO₂/5% O₂. Media were changed on every 2^{nd} day thereafter. Surprisingly, in the presence of a modulator the cells were able to attach to and proliferate on the collagen I-coated surface while maintaining the typical cellular morphology of low cytoplasmic to nuclear ratios but exhibiting an unexpected monolayer, lawn-like growth pattern rather than multilayered colonies. However, without a protectant cell viability was poor (below detectable levels), whereas with the protectant OPH109 by itself the cell morphology changed to larger cytoplasm/nuclear ratios indicating cell differentiation. Blebbistatin as a protectant/modulator was most potent at the lower end of the concentration range (2.5 μM) with the protecting effect being offset by increased toxicity at higher concentrations. This toxic effect could in turn be counteracted by using OPH109 in addition to blebbistatin. After 4-5 days in culture cells were fixed and immunofluorescence stained for the pluripotency markers Oct-3/4, SSEA-4 and Tra-1-81 as well as the early differentiation marker CD29. The cells were imaged and analysed using an IN Cell Analyzer 1000 and IN Cell Developer Toolbox 1.7 software. FIG. 3 summarises the results for 2 cell lines, indicating the ability of protectants/modulators to facilitate feeder-free monolayer culture of undifferentiated hESC. Next, the growth rate of monolayer hESC was assessed by imagining of Hoechst (Invitrogen) stained wells using the IN Cell Analyzer 1000 in 24 hr intervals. Cell number was determined by analysis using IN Cell Developer Toolbox 1.7 and IN Cell Analyzer 1000 Workstation 3.5 software. These experiments revealed a doubling time for of 11 hrs, similar to the growth rate of the inner cell mass in a developing embryo, between 24 hrs and 48 hrs post-plating, and 17 hrs between 48 hrs and 72 hrs post-plating (collagen I coating+Y-27632, FIG. 4). There was no significant difference in cell number or proliferation between uncoated and collagen I coated growth surfaces.

### Example 6

### Use of Monolayer hESC Culture for Pluripotency Assessment

The use of hESC monolayer culture for the assessment of hESC pluripotency was assessed.

Single hESC (Table 1) were generated from either manually-passaged organ culture dishes, collagenase-passaged flasks or flasks from example 4 using 0.05% trypsin/5 mM EDTA as described in Example 5. The cells were plated at a density of 6×10³ per well (˜2×10⁴/cm²) of a collagen I-coated 96-well plate in conditioned KSR medium with 20 ng/ml bFGF and 10 μM Y-27632, and incubated at 37° C./5% CO₂/5% O₂. The medium was changed the following day, with or without Y-27632, then every 2^{nd} day until cells reached a confluency of ˜80%, typically within 3-5 days.

Analysis of pluripotency markers in the monolayer hESC culture was assessed by immunohistochemistry as described above. Imaging revealed that the monolayer hESC cultures retain their expression of pluripotency markers including Nanog, Oct-3/4, SSEA-4, Tra-1-81 and Tra-1-60 and could be used to compare the level of expression between hESC lines (FIG. 5).

### Example 7

### Use of Monolayer hESC Culture for Differentiation Assays

The use of a hESC monolayer culture for differentiation assays was assessed by directed-differentiation to neuronal lineages.

Single hESC were generated using 0.05% trypsin/5 mM EDTA from collagenase passaged hESC as described for Example 4. The cells were plated at a density of 6-9×10³ per well (˜2-3×10⁴/cm²) of a collagen-coated 96-well plate in DMEM-F12 with 1×N2, 1×B27 (both Invitrogen), 100 ng/ml Noggin (R&D) and 10 μM Y-27632, and incubated at 37° C./5% CO₂/5% O₂. The medium was changed the following day, then every 2^{nd} day until cells reached a confluency of ˜80%, typically within 10-12 days. Light microscopy was used to monitor the differentiation process and revealed the loss of pluripotent hESC morphology with differentiating cells becoming smaller and elongated with multiple neurite outgrowths.

Immunohistochemistry for neuronal markers was performed as described above in Example 2, with the following exceptions; primary antibodies Sox2 (R&D systems), Map2 (Sigma), Pax6 (Chemicon) and Tuj1 (Covance) used in combination with secondary antibody anti-mouse IgG Alexa-594 (Invitrogen). Analysis revealed that up to 60% of the differentiated hESC expressed neuronal markers, including more mature markers Map2 and Tuj1.

### Example 8

### Feeder Free Expansion of hESC Using Monolayer Culture

To determine if monolayer hESC culturing could be used to expand hESC feeder free, the expansion capability was assessed, as well as the cells ability to maintain pluripotency during feeder-free monolayer expansion.

Manually passaged and collagenase passaged hESC lines (Table 1) grown on feeder cells were dissociated into single cells using 0.05% trypsin/5 mM EDTA as described in Example 5. Cells were plated into a collagen I coated plates at ˜2×10⁴/cm² in Conditioned KSR medium with 20 ng/ml bFGF and 10 μM Y-27632, and incubated at 37° C./5% CO₂/5% O₂ (passage 1). The medium was changed the following day, then every 2^{nd} day until cells reached ˜80% confluency. Cells were then dissociated into single cells using the 0.05% trypsin/5 mM EDTA protocol, with the exception of incubation with 0.05% trypsin/5 mM EDTA for 3 mins only, counted and plated at ˜2×10⁴/cm² in the above described medium (passage 2). This process was repeated when cells reached confluency (passage 3). At each of the 3 passages, some of the dissociated cells were plated into 96 well plates for pluripotency assessment as described in Example 6. Within 9 days, the theoretical cell number obtained for SIVF019 (collagenase passaged stock) was 24 fold that originally plated (FIG. 6A). Manually passaged hESC SIVF002, SIVF006 and SIVF021 could also be successfully expanded as a monolayer (FIG. 6B).

Immunohistochemistry examining pluripotency markers after 1-3 passages with 0.05% trypsin/5 mM EDTA was performed as described in Example 2. As shown in FIG. 7, SIVF019 hESC which had been passaged 2 and 3 times using 0.05% trypsin/5 mM EDTA retained the expression of pluripotency markers comparable with passage 1.

### Example 9

### Monolayer hESC Culture on Different Surfaces

The attachment and growth of hESC as a monolayer on different surfaces was investigated. This included standard uncoated tissue culture surfaces, and surfaces coated with collagen I and matrigel.

A monolayer of collagenase passaged SIVF019 hESC was prepared using trypsin/EDTA as described in Example 5. Cells were plated in wells of a 96 well plate (BD), either uncoated or coated with collagen I or matrigel (BD). After 6 days in culture, cells were fixed and stained for pluripotency markers as described in Example 2. These experiments showed that in addition to collagen I coated surfaces, hESC can be successfully grown, including maintenance of pluripotency markers, on matrigel and uncoated tissue culture surfaces.

### Example 10

### Karyotyping of Monolayer hESC

The monolayer hESC culture protocol was used to karyotype hESC in situ. Single hESC were generated using 0.05% trypsin/5 mM EDTA from collagenase passaged hESC as described in Example 5. The cells were plated at a density of 2×10⁴/cm² on collagen I or matrigel-coated Thermanox plastic coverslips (Nunc) and grown for ˜48 hr prior to incubation overnight with 0.22 ng/ml colcemid (KaryoMAX) and 37.5 g/ml BrdU in Conditioned KSR medium with 20 ng/ml bFGF. Coverslips were then processed and O-banded using standard protocols. Multiple metaphase cells suitable for karyotyping were present in the prepared samples.

### Example 11

### Transfection of hESC as a Monolayer

The ability to transfect monolayer hESC was investigated.

A monolayer of collagenase passaged SIVF019 hESC was prepared using trypsin/EDTA as described for Example 5. Cells were plated into 96 well plates as described for Example 6, with the exception that 10 μM Y-27632 was maintained in the culture media, and incubated for 3 days prior to transfection. Transfection was performed using Fugene HD reagent (Roche Applied Science) as described by the manufacturer using a DNA to Fugene ratio of 2 μg per 6 μl. Cells were transfected with the equivalent of 200 ng and 350 ng of DNA per cm² growth surface area. The plasmids used for transfection were pESM-nB, pCEP4CY and pUC4.1GnanR expressing blue fluorescent protein directed to the nucleus, a fusion of cyan and yellow fluorescent proteins and green fluorescent protein, respectively. Cells were incubated for a further 3 days prior to fixation and staining with the nuclear dye DRAQ5. The plate was scanned using the IN Cell Analyzer 1000, and analyzed using IN Cell Developer Toolbox 1.7 and IN Cell Analyzer 1000 Workstation 3.5 software. As shown in FIG. 9, the transfection efficiency of monolayer SIVF019 hESC with the plasmids varied from 10-30%, depending on the plasmid and the quantity of transfection regent/DNA mix used.

Although the invention has been described with reference to particular preferred embodiments and examples, it will be understood that variations and modifications in keeping with the spirit and the inventive concept described herein are also within the scope of the invention.

### REFERENCES

Peura, T., A. Bosman, O. Chami, R. P. Jansen, K. Texlova, and T. Stojanov. 2008. Karyotypically normal and abnormal human embryonic stem cell lines derived from PGD-analyzed embryos. Cloning Stein Cells 10: 203-216.

1. A cell culturing medium comprising an agent that modulates myosin II and/or a pathway modulating myosin II capable of protecting cells in culture.

2-4. (canceled)

5. The cell culturing medium according to claim 1, wherein the agent is blebbistatin.

6. The cell culturing medium according to claim 1, wherein the agent is capable of maintaining the cells in a monolayer culture on a surface.

7-9. (canceled)

10. A method of continuously maintaining a culture of cells growing on a first surface, comprising repeated steps of:
(i) detaching the cells from the first surface, and
(ii) culturing said detached cells on a second surface in the cell culturing medium of claim 1.

11. A method of culturing cells in a monolayer on a surface, comprising the step of culturing the cells using the cell culturing medium of claim 1.

12. The method according to claim 11, wherein the cells are cultured in a monolayer on a surface comprising a feeder layer or other surface coating.

13. The method according to claim 12, wherein the feeder layer comprises fibroblasts.

14. The method according to claim 12, wherein the other surface coating is collagen or matrigel.

15. A method of passaging and culturing cells, comprising the steps of:
i. detaching the cells from a first surface; and
ii. culturing the detached cells on a second surface using the cell culturing medium of claim 1.

16. A method of passaging cells between cell culture maintained on a first surface and monolayer cell culture maintained on a second surface, comprising the step of detaching cells from said first surface and culturing said detached cells in a monolayer on said second surface using the cell culturing medium of claim 1.

17. A method of maintaining the cell status between a passage of a cell cultured on a first substrate, comprising the steps of:
(i) detaching the cell from the first surface and
(ii) culturing said detached cell on a second surface using the cell culturing medium of claim 1.

18. A method of maintaining the cell status between a passage of a cell between long-term cell culture maintained on a first surface and short-term expanded monolayer cell culture on a second surface, comprising the steps of:
(i) detaching the cell from the first surface, and
(ii) culturing said detached cell on a second surface using the cell culturing medium of claim 1.

19. The method according to claim 15, wherein the first and/or second surface comprises a feeder layer.

20. The method according to claim 1, wherein the feeder layer comprises fibroblasts.

21. The method according to claim 15, wherein the first and/or second surface comprises a coating of collagen or matrigel.

22. The method according to claim 10, wherein the cells are detached from the first surface using a cell passaging medium comprising at least one agent capable of detaching from a surface a cell that is cultured in vitro on said surface, wherein the agent capable of detaching cells from a surface is metal ion chelating agent and/or a proteolytic enzyme.

23. The method according to claim 22, wherein the passaging medium further comprises a water-soluble polymer selected from the group consisting of gelatin, polyvinylpyrrolidone (PVP), polyethylene glycol (PEG), agarose, dextran, polypeptides, polysaccharides and polynucleotides.

24-30. (canceled)

31. A cell cultured by a method according to claim 10.

32. The method according to claim 10, wherein the cell is a mammalian cell.

33. The method according to claim 32, wherein the mammalian cell is a stem cell.

34. The method according to claim 33, wherein the stem cell is a pluripotent stem cell.

35. The method according to claim 34, wherein the pluripotent stem cell is a human pluripotent embryonic stem cell or a human induced pluripotent stem cell.
